(19) United States
(12) Patent Application Publication
(10) Pub. No.: US 2025/0223562 A1
(43) Pub. Date: Jul. 10, 2025

(54) IMMUNOSELECTED UMBILICAL CORD-DERIVED MESENCHYMAL STEM CELLS (UC-MSCs) AND DOWNSTREAM BIOLOGICS

(71) Applicant: GLOBUS MEDICAL, INC., AUDUBON, PA (US)

(72) Inventors: Bipin Gaihre, Spring City, PA (US); Archana Bhat, Phoenixville, PA (US); Joshua Cige, Conshohocken, PA (US); Amit Vora, Philadelphia, PA (US)

(21) Appl. No.: 18/406,302

(22) Filed: Jan. 8, 2024

(51) Int. Cl. C12N 5/0775; A61K 35/28

(52) U.S. Cl. CPC C12N 5/0667; A61K 35/28; C12N 2500/02; C12N 2501/71

(57) ABSTRACT

Disclosed herein are methods for isolation and expansion of umbilical cord-derived mesenchymal stem cells (UC-MSCs) having one or more cell surface markers, and compositions comprising such UC-MSCs. Also disclosed herein are further processing methods for use in connection with the UC-MSCs to isolate extracellular vesicles (EVs) therefrom. Also disclosed herein are lyophilized EVs isolated from the UC-MSCs, and adapted for use as a therapeutic product.

### FIELD OF THE DISCLOSURE

Various embodiments of the disclosure relate to isolation and expansion of biologic therapeutics and downstream processing thereof. More particularly, the disclosure relates to the isolation and expansion of umbilical cord-derived mesenchymal stem cells (UC-MSCs), and their use in the preparation of biologic therapeutic products for use in the treatment of degenerative musculoskeletal diseases.

### INTRODUCTION

Degenerative musculoskeletal diseases (DMDs) are a leading cause of pain-associated functional decline, resulting in long-term debilitation. Current treatment strategies for a cluster of DMDs including osteoarthritis, degenerative disc disease (DDD), and osteoporosis focus on relieving pain through conventional and conservative treatments, such as physical therapy and over-the-counter pain medications. In many cases, these treatments fail to provide consistent pain relief, leading to the use of opioids and other epidural injections to manage sufferers' pain. Many of these pain management strategies also have high failure rates, leading a significant portion of the patient population to seek surgical solutions.

Better therapeutics for early intervention are needed to relieve pain and to delay or avoid entirely the need for surgery through tissue repair and regenerative actions. Mesenchymal stem cell (MSC)-based therapies have emerged as attractive candidates for such applications, owing to their unique properties related to mechanism of action and effects on tissue regeneration. MSCs are multi-potent cells, capable of self-renewal and differentiation into a number of different cell types within the mesenchymal lineage. Such cell types include, inter alia, osteoblasts, chondrocytes, myocytes, and adipocytes.

Allogenic MSC therapy involves isolation of MSCs from donor tissues, expansion in vitro, and using the expanded cells as a therapeutic drug. Active processing during the in vitro expansion of MSCs contributes to achievement of clinically meaningful cell numbers for regenerative therapy applications. In particular, immunoselection of MSC populations is useful to achieve purified subset populations that express specific cell surface antigens or specific proteins during in vitro expansion, resulting in improved therapeutic outcomes due to, e.g., increased MSC survival rates, reduced MSC apoptotic rates when introduced into a host treatment site, improved engraftment of allogenic cells to the host tissue, and paracrine signaling to promote regenerative activities of the host cells. Immunoselection of MSC populations is also useful for the creation of downstream therapeutic products offering unique attributes.

### SUMMARY

A first aspect of the disclosure provides a method comprising isolating umbilical cord-derived mesenchymal stem cells (UC-MSCs) from perivascular Wharton's Jelly from a human umbilical cord; and expanding the UC-MSCs, wherein the isolating and the expanding are performed a) under hypoxic conditions, or b) in the presence of a prolyl hydroxylase (PHD) enzyme inhibitor, or c) under hypoxic conditions and in the presence of the prolyl hydroxylase (PHD) enzyme inhibitor.

Various embodiments of the method may additionally or alternatively include one or more of the following features: the hypoxic conditions comprise an oxygen concentration of about 1% to about 10%; the PHD enzyme inhibitor comprises Roxadustat, and the Roxadustat is present at a concentration of about 0.1 μg/mL to about 100 μg/mL during the isolation and the expansion.

In certain embodiments, at least 50% of the derived UC-MSCs are positive for: a) a cell surface marker selected from CD73, CD90, and CD105, b) a cell surface marker selected from CD166 and HLA-ABC, c) both of a cell surface marker selected from CD73, CD90, and CD105, and a cell surface marker selected from CD166 and HLA-ABC, or d) any combination of two or more of the foregoing cell surface markers.

In certain embodiments, under chondrogenic and hypoxic environment, UC-MSC population will have the capability to synthesize an extracellular matrix (ECM) component, wherein the ECM component could be sulphated glycosaminoglycans (sGAG) or collagen II (ColII) or both.

In certain embodiments, the method further includes using fluorescence-assisted cell sorting (FACS) to derive a subset UC-MSC population that is positive for HIF-1α. In particular, at least 90% of the subset UC-MSC population is positive for HIF-1α, and at least 50% of the subset UC-MSC population is also positive for: a) a cell surface marker selected from CD73, CD90, and CD105, b) a cell surface marker selected from CD166 and HLA-ABC, or c) both of a cell surface marker selected from CD73, CD90, and CD105, and a cell surface marker selected from CD166 and HLA-ABC.

In certain embodiments, the method further comprises processing the derived UC-MSC population to isolate extracellular vesicles (EVs). The processing comprises performing differential ultracentrifugation to produce a heterogeneous population of EVs, wherein the differential ultracentrifugation is performed at a relative centrifugal force (RCF) or g force of about 300 g to about 200,000 g, and for a duration of time from about 5 minutes to about 120 minutes. In certain embodiments, the method may further comprise lyophilizing the heterogeneous population of EVs to obtain a dry powder; and storing the dry powder in a vial at a temperature of about 0° C. to about 4° C.

In certain embodiments, the method further comprises reconstituting the dry powder with saline solution to yield a therapeutic product. The therapeutic product is adapted for use in the treatment of degenerative musculoskeletal diseases in a patient in need thereof.

A second aspect of the invention provides a composition comprising extracellular vesicles (EVs) derived from umbilical cord-derived mesenchymal stem cells (UC-MSCs), the composition being adapted for use as a therapeutic product.

Various embodiments of the composition may additionally or alternatively include one or more of the following features: the composition further comprises UC-MSCs capable of secreting sulphated glycosaminoglycans (sGAG), Collagen II, and interleukin 1 receptor antagonist (IL1Ra) protein; and the UC-MSCs are immunoselected by treatment with a prolyl hydroxylase (PHD) enzyme inhibitor and/or exposure to hypoxic conditions during isolation and expansion of the UC-MSCs. The UC-MSCs may be immunoselected on the basis of being positive for HIF-1α.

In certain embodiments, the EVs are lyophilized to obtain a dry powder, the dry powder being adapted for reconstitution with saline solution to form a therapeutic product. The therapeutic product is adapted for use in the treatment of degenerative musculoskeletal diseases in a patient in need thereof.

In certain embodiments, the composition is prepared by a process comprising: isolating umbilical cord-derived mesenchymal stem cells (UC-MSCs) from perivascular Wharton's Jelly from a human umbilical cord; expanding the UC-MSCs, wherein the isolating and the expanding are performed a) under hypoxic conditions, or b) in the presence of a prolyl hydroxylase (PHD) enzyme inhibitor, or c) under hypoxic conditions and in the presence of the prolyl hydroxylase (PHD) enzyme inhibitor; and isolating and lyophilizing the EVs.

A third aspect of the disclosure provides a product comprising a UC-MSC population, in which at least 50% of the derived cells are positive for: a cell surface marker selected from CD73, CD90, and CD105, a cell surface marker selected from CD166 and HLA-ABC, or a combination of two or more of the foregoing. The population may be prepared according to the methods described herein.

A fourth aspect of the disclosure provides a product comprising a purified UC-MSC subset population, in which at least 50% of the derived cells are positive for: a cell surface marker selected from CD73, CD90, and CD105, a cell surface marker selected from CD166 and HLA-ABC, or a combination of two or more of the foregoing, and at least 90% of the derived cells are positive for HIF-1α. The population may be prepared according to the methods described herein.

These and other aspects, advantages, and salient features of the disclosure will become apparent from the following detailed description, which, when taken in conjunction with the annexed drawings, disclose embodiments of the invention.

### BRIEF DESCRIPTION OF THE DRAWINGS

The accompanying drawings, which are incorporated into and constitute a part of this specification, illustrate various exemplary embodiments and, together with the description, serve to explain the principles of the disclosed embodiments. The drawings show different aspects of the present disclosure. Where appropriate, reference numerals illustrating like structures, components, materials, and/or elements in different figures are labeled similarly. It is understood that various combinations of the structures, components, and/or elements, other than those specifically shown, are contemplated and are within the scope of the present disclosure.

The embodiments described herein are not limited to any combinations and/or permutations of such aspects and/or embodiments. Moreover, each of the aspects of the described inventions, and/or embodiments thereof, may be employed alone or in combination with one or more of the other aspects of the described inventions and/or embodiments thereof. For the sake of brevity, certain permutations and combinations are not discussed and/or illustrated separately herein. Notably, an embodiment or implementation described herein as “exemplary” is not to be construed as preferred or advantageous, for example, over other embodiments or implementations; rather, it is intended reflect or indicate the embodiment(s) is/are “example” embodiment(s).

FIG. 1 provides a flow chart depicting a method for generating a UC-MSC population as described herein, in an accordance with an embodiment of the disclosure.

FIG. 2 provides a flow chart depicting a method for downstream processing of derived UC-MSCs as described herein, in an accordance with an embodiment of the disclosure.

FIGS. 3A-3C illustrate UC-MSC isolation and expansion under hypoxic conditions, in the presence of Roxadustat, or a combination thereof, according to embodiments of the disclosure.

FIGS. 4A-4D illustrate flow panels for MSC-specific surface markers for cells isolated and expanded under hypoxic conditions.

FIG. 5 illustrates sGAG synthesis, under chondrogenic and hypoxic condition, (quantified in μg of sGAG per mg of dry weight) by UC-MSCs isolated and expanded under hypoxic conditions.

FIG. 6 illustrates schematics for exosome derivation from UC-MSCs.

It is noted that the drawings of the disclosure are not necessarily to scale.

### DETAILED DESCRIPTION

Embodiments of the present disclosure relate to isolation of biologic therapeutics including immunoselected umbilical cord-derived mesenchymal stem cells (UC-MSCs), and therapeutic products derived therefrom.

As used herein, the terms “comprises,” “comprising,” “includes,” “including,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” In addition, the terms “first,” “second,” and the like, herein do not denote any order, quantity, or importance, but rather are used to distinguish an element or a structure from another. Moreover, the terms “a” and “an” herein do not denote a limitation of quantity, but rather denote the presence of one or more of the referenced items. Further, as used herein, the terms “about,” “substantially,” and “approximately” generally mean±10% of the indicated value.

As used herein, the term “subject” refers to an animal on whom a procedure is to be performed. The animal may be a mammal, and may more particularly be a human. The terms “subject,” “patient,” and “host” may be used interchangeably herein. The terms “clinician” and “user” may be used interchangeably to refer to the individual performing the administration of the procedure on the subject as described herein.

As previously discussed, isolation of biologic therapeutics including immunoselected umbilical cord-derived mesenchymal stem cells (UC-MSCs), and therapeutic products derived therefrom are disclosed herein. The UC-MSCs may include, inter alia, hypoxia-inducible factor 1 alpha (HIF-1α)-immunoselected UC-MSCs. Hypoxia inducible factor (HIF) is a heterodimeric complex of HIF α and β subunits, which under hypoxic conditions, bind to a core sequence of the hypoxia response element (HRE) of target genes to activate the downstream signaling cascade. Some of the target genes include, inter alia, SOX-9, matrix metalloproteinase (MMP), and bone morphogenetic protein (BMP). However, under normoxic conditions, the HIF-1a protein is unstable; prolyl hydroxylase (PHD) hydroxylates the a subunit that leads to its protease degradation. HIF is believed to directly or indirectly regulate more than one hundred genes, including genes that regulate apoptosis and cellular proliferation. Cells that express HIF-1α are more resistant to apoptosis, and have a higher proliferative capacity under a hypoxic and glucose-deprived microenvironment.

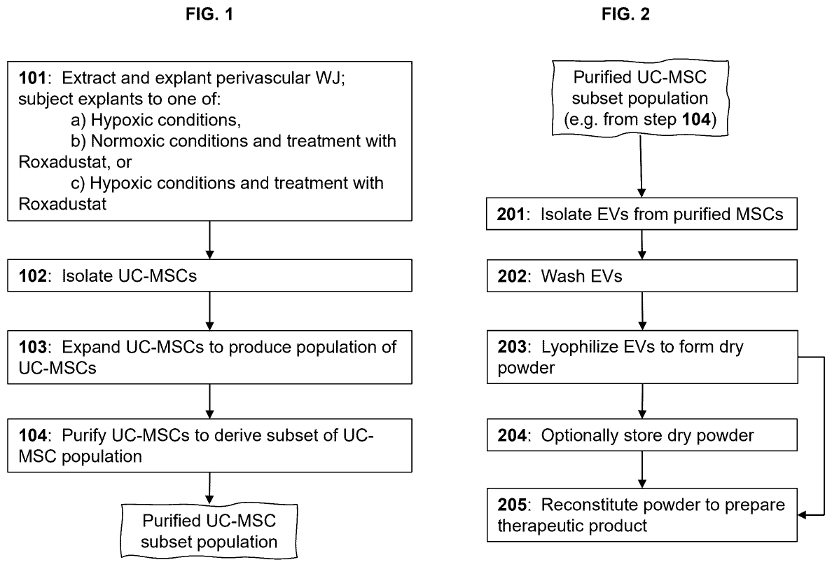

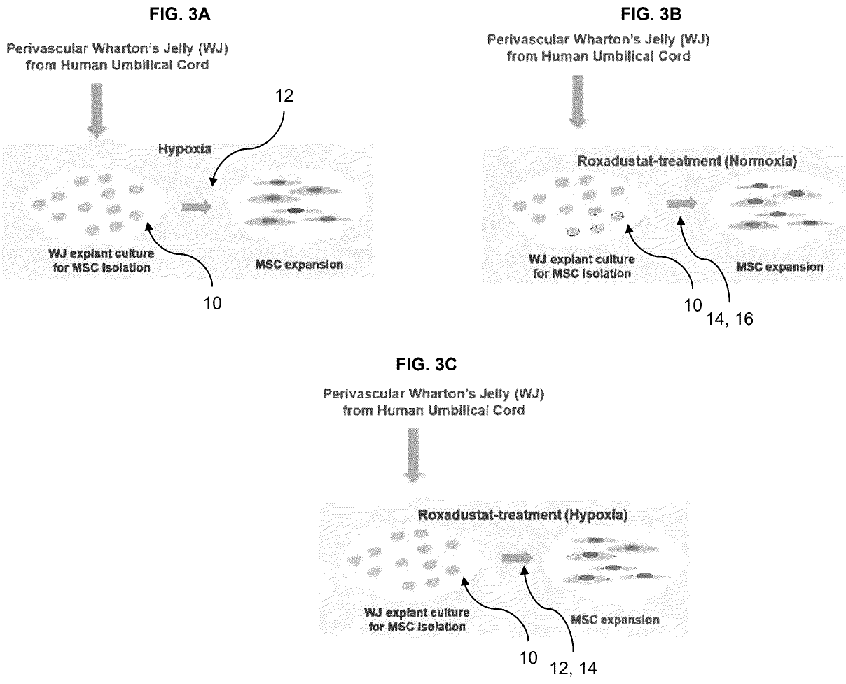

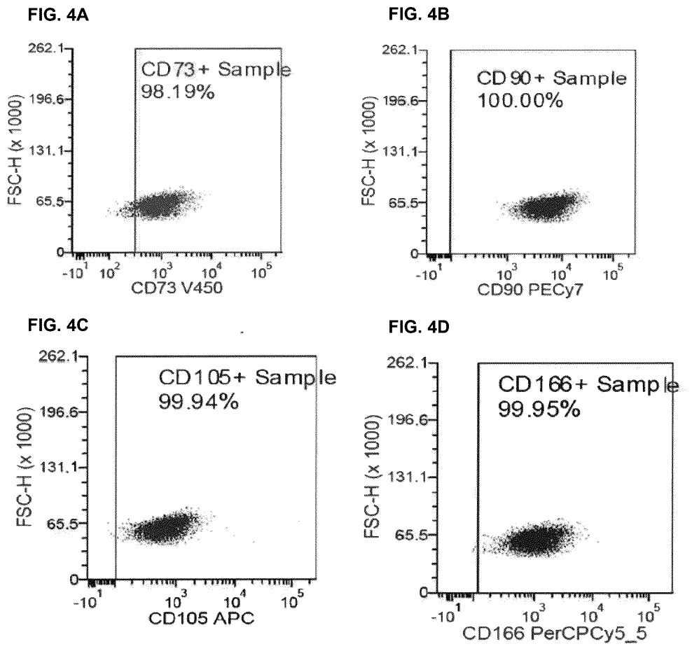

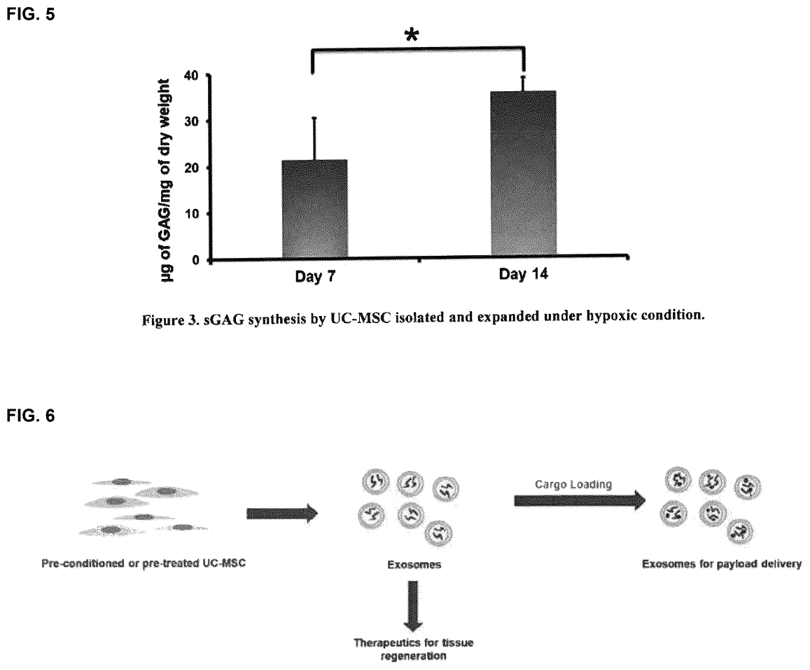

As discussed above, and with reference to FIG. 1, embodiments of the disclosure provide methods for generating a UC-MSC population immunoselected to demonstrate particular characteristics, as described herein. In the method of FIG. 1, in step 101, human umbilical cord tissue is dissected to extract the perivascular Wharton's Jelly (WJ). Explants of the perivascular WJ may be placed on a tissue culture flask with a growth medium, and placed inside an oxygen-controlled incubation chamber. Conditions within the incubation chamber may vary as described herein and as depicted in FIGS. 3A-3C.

Referring to FIG. 3A, the explants 10 may be subjected to hypoxic conditions 12, which may include an oxygen concentration within the incubation chamber of about 1% to about 10%, or more particularly, about 3%. The concentrations of carbon dioxide and nitrogen may be about 5% and about 92%, respectively. In the embodiment of FIG. 3B, the explants 10 may be supplemented with a prolyl hydroxylase (PHD) enzyme inhibitor 14, and placed in the incubation chamber under normoxic conditions 16, e.g., at an oxygen concentration of about 21%. The concentrations of carbon dioxide and nitrogen may be about 5% and atmospheric condition, respectively. The PHD enzyme inhibitor 14 may include Roxadustat (FG-4592), which may be present at a concentration of about 0.1 μg/mL to about 100 μg/mL, or more particularly about 10 μg/mL. In the embodiment of FIG. 3C, the explants 10 may be supplemented with a prolyl hydroxylase (PHD) enzyme inhibitor 14, and placed in the incubation chamber under hypoxic conditions 12. In particular, the PHD enzyme inhibitor 14 may include Roxadustat, which may be present at a concentration of about 0.1 μg/mL to about 100 μg/mL, or more particularly about 10 μg/mL, and the hypoxic conditions 12 may include an oxygen concentration within the incubation chamber of about 1% to about 10%, or more particularly, about 3%, and carbon dioxide and nitrogen concentrations of about 5% and about 92%, respectively.

Referring back to FIG. 1, regardless of which of the foregoing conditions are deployed in the incubation chamber, a period of about seven (7) to about fourteen (14) days is allowed to elapse, following which period the UC-MSCs attached to the flask are isolated in step 102. The isolated UC-MSCs then undergo expansion in step 103 under the same conditions as in the preceding period of time. The expansion of step 103 occurs during an additional time period of, e.g., about three (3) to about fifteen (15) days, allowing for additional generation of UC-MSCs.

The isolation and expansion steps 102, 103 may produce a population of UC-MSCs having particular attributes or cell surface markers. These markers may include, e.g., cluster of differentiation (CD) molecules, HLA molecules, and other cell surface markers. For example, the foregoing isolation and expansion steps may produce a population of UC-MSCs in which at least 50% of the UC-MSCs are positive for: a) a cell surface marker selected from CD73, CD90, and CD105; b) a cell surface marker selected from CD166 and HLA-ABC, c) both of a cell surface marker selected from CD73, CD90, and CD105, and a cell surface marker selected from CD166 and HLA-ABC; or d) any other combination of the foregoing cell surface markers. Referring to FIGS. 4A-4D, the flow panels illustrate the presence of MSC-specific surface markers for UC-MSCs isolated and expanded under hypoxic conditions. More particularly, the samples show positivity for exemplary cell surface markers CD73 (98.19%), CD90 (100.00%), CD105 (99.94%), and CD166 (99.95%).

Referring back to FIG. 1, a further embodiment includes an additional step 104 to purify the UC-MSC population prepared in step 103, e.g., by selecting a subset population of UC-MSCs based on the presence of a particular cell surface marker. For example, fluorescence assisted cell sorting (FACS) may be used in step 104 to derive a subset population of UC-MSCs from the population of step 103. FACS may be used to sort and select for UC-MSCs on the basis of, e.g., positivity for the presence of HIF-1α. In one embodiment, at least 90% of the subset UC-MSC population is positive for HIF-1α, and at least 50% of the subset UC-MSC population is also positive for a) a cell surface marker selected from CD73, CD90, and CD105; b) a cell surface marker selected from CD166 and HLA-ABC, c) both of a cell surface marker selected from CD73, CD90, and CD105, and a cell surface marker selected from CD166 and HLA-ABC; or d) any other combination of the foregoing cell surface markers, as in the underlying UC-MSC population derived in step 103.

The UC-MSCs derived according to the foregoing steps may have the ability to synthesize major extracellular matrix (ECM) components such as, e.g., sulphated glycosaminoglycans (sGAG) and collagen II when subjected to chondrogenic differentiation medium under hypoxic condition. Thus, the methods described herein may further include inducing the UC-MSCs to synthesize an extracellular matrix (ECM) component such as sGAG. The ECM component synthesis may increase over time, such that a greater amount of sGAG is synthesized at day fourteen (14) relative to day seven (7). The UC-MSCs derived according to the foregoing steps may further have the ability to secrete anti-inflammatory cytokines, under hypoxic condition, such as, e.g., interleukin-1 receptor antagonist (ILIRa) protein. IL1Ra binds with IL-1, a major proinflammatory factor, in the degenerative cascade in the spine. This prevents activation of IL-1, thereby reducing inflammation, which in turn reduces pain associated with DMD.

As discussed above, and with reference to FIG. 2, embodiments of the disclosure provide methods for downstream processing of UC-MSCs to generate a specific extracellular vesicle (EV) as described herein. The process of FIG. 2 may be performed on the UC-MSCs derived according to the methods described above and depicted in the flow chart of FIG. 1. As such, the methods depicted in FIG. 1 and FIG. 2 may be understood to form a single method, including both generation of a UC-MSC population as described herein and downstream processing thereof. However, the use of the purified UC-MSC subset population derived in step 104 of FIG. 1 in the process of FIG. 2 is merely an exemplary population. In other embodiments, the methods of FIG. 1 and FIG. 2 may be performed independently of one another: the process of FIG. 1 may be performed in the absence of the process of FIG. 2, and the process of FIG. 2 may be performed on a cell population other than those derived through steps 101, 102, 103, and/or 104 of FIG. 1.

As shown in FIG. 2, the process begins with a UC-MSC population which may be obtained, e.g., using the methods described relative to FIG. 1. The UC-MSCs may then be processed to isolate extracellular vesicles (EVs) therefrom in step 201. The isolating of step 201 may include performing differential ultracentrifugation on lysed UC-MSCs to produce a heterogeneous population of EVs. In various embodiments, the differential ultracentrifugation may be performed by subjecting the lysate to repeated centrifugations at increasing centrifugal forces and/or durations of time. After each centrifugation, the supernatant may be removed from the tube, and re-centrifuged at an increased centrifugal force. Centrifugal forces may be in a range of, e.g., from about 300 g to about 200,000 g, and may be applied for a duration of time from, e.g., about five (5) minutes to about one hundred twenty (120) minutes. One exemplary method may include subjecting lysed UC-MSCs to differential ultracentrifugation with the following example scheme: about fifteen (15) minutes at 300 g; followed by about five (5) minutes at 5,000 g; followed by about fifteen (15) minutes at 20,000 g; followed by filtration through a filter having, e.g., a 400 μm membrane filter; followed by about sixty (60) minutes at 200,000 g.

In step 202, after completing differential ultracentrifugation, the resulting EVs may be washed. In step 203, the heterogeneous population of EVs may be lyophilized to obtain a dry powder that is rich in anti-inflammatory cytokines, signaling molecules, and proteins. In step 204, the powder may optionally be stored in a vial at a temperature of, e.g., about 0° C. to about 4° C. In step 205, the lyophilized powder, either of step 203 or as stored in step 204, may be reconstituted to yield a therapeutic product. The powder may be reconstituted at a concentration of, e.g., about 10 to 100 μg per mL of a solution such as, e.g., saline solution. The reconstituted powder may be used as a therapeutic product which may be adapted for use as an injectable therapy, offering the advantages of longer-term outcomes than existing injections for degenerative musculoskeletal diseases, without the drawbacks of opioids.

In another embodiment, a product is provided comprising a UC-MSC population, in which at least 50% of the derived cells are positive for: a cell surface marker selected from CD73, CD90, and CD105, a cell surface marker selected from CD166 and HLA-ABC, or a combination of two or more of the foregoing. The population may be prepared according to the methods described herein, with reference to FIG. 1.

In another embodiment, a product is provided comprising a purified UC-MSC subset population, in which at least 50% of the derived cells are positive for: a cell surface marker selected from CD73, CD90, and CD105, a cell surface marker selected from CD166 and HLA-ABC, or a combination of two or more of the foregoing, and at least 90% of the derived cells are positive for HIF-1α. The population may be prepared according to the methods described herein, with reference to FIG. 1.

According to a further embodiment, a composition is provided herein, comprising extracellular vesicles (EVs) derived from umbilical cord-derived mesenchymal stem cells (UC-MSCs). The composition may be adapted for use as a therapeutic product.

The composition may be prepared by a process comprising isolating umbilical cord-derived mesenchymal stem cells (UC-MSCs) from perivascular Wharton's Jelly from a human umbilical cord, and expanding the UC-MSCs under conditions that are a) hypoxic, b) normoxic and in the presence of a prolyl hydroxylase (PHD) enzyme inhibitor, or c) hypoxic and in the presence of a PHD enzyme inhibitor. Following expansion, the EVs may be isolated from the UC-MSCs.

In various embodiments, the composition may further comprise UC-MSCs capable of synthesizing one or more of sulphated glycosaminoglycans (sGAG), Collagen II, and secreting interleukin 1 receptor antagonist (ILIRa) protein, under hypoxic condition. In various embodiments, the UC-MSCs from which the EVs are derived may be HIF-1α immunoselected by treatment with a prolyl hydroxylase (PHD) enzyme inhibitor or incubation under hypoxic conditions.

The EVs may be lyophilized to obtain a dry powder that is storable, e.g., in vials at a temperature of about 0° C. to about 4° C., and is adapted for reconstitution with to form a therapeutic product either after or in lieu of such storage. When desired, the powder may be reconstituted at a concentration of, e.g., about 10 to 100 μg per mL of a solution such as, e.g., a saline solution. The reconstituted powder may be used as a therapeutic product, e.g., for tissue regeneration. In other embodiments, the lyophilized EVs may be used as cargo vehicles for the delivery of drug molecules or signaling molecules to a target site in a patient. Exemplary molecules include, e.g., compounds that inhibit growth of Propionibacterium acnes (P. acnes), Cutibacterium acnes (C. acnes), or both.

### EXAMPLES

### Example 1

Human umbilical cord tissue is dissected to extract the perivascular WJ therein. The perivascular WJ is explanted on a tissue culture flask with a growth medium, and placed inside an oxygen-controlled incubation chamber with oxygen concentration set to 3%, carbon dioxide concentration set to 5%, and nitrogen concentration set to 92%. After about seven (7) days to about fourteen (14) days, the adherent UC-MSCs are isolated and expanded under the same conditions for an additional period of about three (3) days to about fifteen (15) days. In the resulting UC-MSC population, at least 50% of the derived cells are positive for: a cell surface marker selected from CD73, CD90, and CD105, a cell surface marker selected from CD166 and HLA-ABC, or a combination of two or more of the foregoing (see FIGS. 4A-4D).

### Example 2

Human umbilical cord tissue is dissected to extract the perivascular WJ therein. The perivascular WJ is explanted on a tissue culture flask with a growth medium supplemented with 10 μg/mL of Roxadustat, and placed inside an oxygen-controlled incubation chamber. The chamber may either be set to hypoxic conditions, in which the oxygen concentration is set to 3%, carbon dioxide concentration set to 5%, and nitrogen concentration set to 92%; or normoxic conditions, in which the oxygen concentration is set to 21%, carbon dioxide concentration set to 5%, and nitrogen concentration set to atmospheric conditions. After about seven (7) days to about fourteen (14), the adherent UC-MSCs are isolated and expanded under the same conditions for an additional period of about three (3) days to about fifteen (15) days. In the resulting UC-MSC population, at least 50% of the derived cells are positive for: a cell surface marker selected from CD73, CD90, and CD105, a cell surface marker selected from CD166 and HLA-ABC, or a combination of two or more of the foregoing.

### Example 3

The UC-MSC population derived in Example 1 or Example 2 is further purified using FACS to prepare a subset UC-MSC population selected based on the presence of HIF-1α. At least 50% of the derived cells are positive for: a cell surface marker selected from CD73, CD90, and CD105, a cell surface marker selected from CD166 and HLA-ABC, or a combination of two or more of the foregoing. Additionally, at least 90% of these cells are HIF-1α positive.

### Example 4

The UC-MSC population derived in Example 1 or Example 2 synthesizes ECM components such as, e.g., sGAG, when subjected to chondrogenic differentiation medium, under hypoxic condition. The amount of sGAG in μg of sGAG/mg of dry weight secreted at day fourteen (14) is greater than the amount of sGAG secreted at day seven (7) (see FIG. 5).

### Example 5

The UC-MSC population derived in Example 1 or Example 2 secretes anti-inflammatory cytokines such as, e.g., the IL1Ra protein, under hypoxic condition.

### Example 6

The UC-MSC population derived in Example 1 or Example 2 is further processed to isolate the EVs as illustrated in FIG. 6. The EVs are isolated using established techniques such as, e.g., differential ultracentrifugation. UC-MSCs are subjected to differential ultracentrifugation according to the following scheme: relative centrifugal force of 300 g for fifteen (15) minutes; 5,000 g for five (5) minutes; 20,000 g for fifteen (15) minutes; filtration through a 400 μm membrane filter; and 200,000 g for sixty (60) minutes. The resulting composition is washed and lyophilized to a dry powder of EVs.

### Example 7

The lyophilized EVs powder prepared in Example 6 is reconstituted at 10 to 100 μg/mL of saline solution, and used as a therapeutic product for tissue regeneration for the treatment of degenerative musculoskeletal diseases.

### Example 8

The lyophilized EVs prepared in Example 6 are used as cargo vehicles for the delivery of compounds that inhibit growth of Propionibacterium acnes (P. acnes), Cutibacterium acnes (C. acnes), or both.

While various embodiments are described herein, it will be appreciated from the specification that various combinations of elements, variations or improvements therein may be made by those skilled in the art, and are within the scope of the invention. In addition, many modifications may be made to adapt a particular situation or material to the teachings of the invention without departing from essential scope thereof. Therefore, it is intended that the invention not be limited to the particular embodiment disclosed as the best mode contemplated for carrying out this invention, but that the invention will include all embodiments falling within the scope of the appended claims.

What is claimed is:

1. A method comprising:
isolating umbilical cord-derived mesenchymal stem cells (UC-MSCs) from perivascular Wharton's Jelly from a human umbilical cord; and
expanding the UC-MSCs to derive a population of expanded UC-MSCs, wherein the isolating and the expanding are performed:
a) under hypoxic conditions, or
b) in the presence of a prolyl hydroxylase (PHD) enzyme inhibitor, or
c) under hypoxic conditions and in the presence of the prolyl hydroxylase (PHD) enzyme inhibitor.

2. The method of claim 1, wherein the hypoxic conditions comprise an oxygen concentration of about 1% to about 10%.

3. The method of claim 1, wherein the PHD enzyme inhibitor comprises Roxadustat.

4. The method of claim 3, wherein the Roxadustat is present at a concentration of about 0.1 μg/mL to about 100 μg/mL during the isolation and the expansion.

5. The method of claim 1, wherein at least 50% of the population of expanded UC-MSCs are positive for:
a) a cell surface marker selected from CD73, CD90, and CD105,
b) a cell surface marker selected from CD166 and HLA-ABC, or
c) both of the cell surface marker selected from CD73, CD90, and CD105, and the cell surface marker selected from CD166 and HLA-ABC.

6. The method of claim 1, further comprising:
using fluorescence-assisted cell sorting (FACS), deriving a subset UC-MSC population from the population of expanded UC-MSCs that is positive for HIF-1α.

7. The method of claim 6, wherein at least 90% of the subset UC-MSC population is positive for HIF-1α, and at least 50% of the subset UC-MSC population is positive for:
a) a cell surface marker selected from CD73, CD90, and CD105,
b) a cell surface marker selected from CD166 and HLA-ABC, or
c) both of the cell surface marker selected from CD73, CD90, and CD105, and the cell surface marker selected from CD166 and HLA-ABC.

8. The method of claim 6, further comprising processing the subset UC-MSC population to isolate extracellular vesicles (EVs).

9. The method of claim 8, wherein isolating the EVs further comprises performing differential ultracentrifugation to produce a heterogeneous population of EVs.

10. The method of claim 9, wherein the differential ultracentrifugation is performed at a relative centrifugal force of about 300 g to about 200,000 g, and for a duration of time from about 5 minutes to about 120 minutes.

11. The method of claim 9, further comprising lyophilizing the heterogeneous population of EVs to obtain a dry powder; and storing the dry powder in a vial at a temperature of about 0° C. to about 4° C.

12. The method of claim 11, further comprising reconstituting the dry powder with saline solution, to yield a therapeutic product.

13. The method of claim 1, further comprising:
subjecting the UC-MSCs to chondrogenic differentiation medium under hypoxic condition, thereby inducing the UC-MSCs to synthesize an extracellular matrix (ECM) component.

14. The method of claim 13, wherein the ECM component is sulphated glycosaminoglycans (sGAG) or Collagen II or both.

15. A composition comprising:
extracellular vesicles (EVs) derived from umbilical cord-derived mesenchymal stem cells (UC-MSCs),
the composition being adapted for use as a therapeutic product.

16. The composition of claim 15, further comprising of: interleukin 1 receptor antagonist (IL1Ra) protein.

17. The composition of claim 15, wherein the UC-MSCs are HIF-1α immunoselected by treatment with a prolyl hydroxylase (PHD) enzyme inhibitor.

18. The composition of claim 15, wherein the EVs are lyophilized to obtain a dry powder, the dry powder being adapted for reconstitution with saline solution to form the therapeutic product.

19. The composition of claim 15, prepared by a process comprising:
isolating umbilical cord-derived mesenchymal stem cells (UC-MSCs) from perivascular Wharton's Jelly from a human umbilical cord;
expanding the UC-MSCs,
wherein the isolating and the expanding are performed:
a) under hypoxic conditions, or
b) in the presence of a prolyl hydroxylase (PHD) enzyme inhibitor, or
c) under hypoxic conditions and in the presence of the prolyl hydroxylase (PHD) enzyme inhibitor; and
isolating and lyophilizing the EVs.

20. A composition comprising:
a population of isolated and expanded umbilical cord-derived mesenchymal stem cells (UC-MSCs),
wherein at least 90% of the UC-MSCs in the population are positive for HIF-1α, and at least 50% of the UC-MSCs in the population are positive for:
a) a cell surface marker selected from CD73, CD90, and CD105,
b) a cell surface marker selected from CD166 and HLA-ABC, or
c) both of the cell surface marker selected from CD73, CD90, and CD105, and the cell surface marker selected from CD166 and HLA-ABC.
